(19) United States
(12) Patent Application Publication
(10) Pub. No.: US 2004/0024534 A1
(43) Pub. Date: Feb. 5, 2004

(54) Process of creating an index for diagnosis or prognosis purpose

(75) Inventor: Ching-Hsiang Hsu, Tainan County (TW)

Correspondence Address: BANNER & WITCOFF, 1001 G STREET N W, SUITE 1100, WASHINGTON, DC, 20001, US

(73) Assignee: TaiMont Biotech Inc., Tainan County (TW)

(21) Appl. No.: 10/210,086

(22) Filed: Aug. 2, 2002

(51) Int. Cl. C12Q 1/68; G06F 19/00; G01N 33/48; G01N 33/50; C12P 19/34

(52) U.S. Cl. 702/20; 435/6; 435/91.2

(57) ABSTRACT

The present invention mainly relates to a process of creating an index for diagnosis and/or prognosis of a complex disease trait by using a correlation formula obtained by the statistic analysis and regression process for condition scores and the expression values of the gene selected to be related to the complex disease trait. A process of creating an asthma index for diagnosis and/or prognosis of asthma is also provided in the invention.

### BACKGROUND OF THE INVENTION

1. Field of the Invention

The invention mainly relates to a process for creating an index for diagnosis and/or prognosis of a complex disease trait, such as asthma.

2. Description of the Related Art

Genomic medicine can be defined as the use of genotypic analysis to enhance the quality of medicine care, including pre-symptomatic identification to disease, preventive intervention, selection of pharmacotherapy, and individual design of medical care based on genotype. Genomic medicine gains increasing importance due to a fast development in the human genomics and molecular medicine. Nowadays, genotypic analysis becomes a standard practice for diagnosis or treatment of a disease in which a single gene plays a prominent role. By contrast, genotypic analysis has not yet been used in diagnosis or treatment of a complex disease trait in which multiple genes and non-genetic factors are involved.

It is believed that a complex disease trait, also known as a multifactorial disease, is related to multiple genes, non-genetic factors, and the interaction between the multiple genes and non-genetic factors. For example, type 1, or insulin-dependent, diabetes has been reported to be related to at least 10 genes, including the HLA region and the insulin gene, but not a single gene.

It has been reported that asthma is related to many genes (Joos L and Stanford A J, Genotype predictors of response to asthma medications. Current Opinion in Pulmonary Medicine 2002;8:9-15; Quinzii C et al., Predictive genetic testing-new possibilities in determination of risk of complex diseases. Croatian Medical Journal. 2001;42(4):458-462). The genes related to asthma are capable of regulating the balance of cytokines of Th1 and Th2 cells (Rogge L et al., Transcript imaging of the development of human T helper cells using oligonucleotide arrays. Nat Genet. 2000;25(1):96-101). Besides, the non-genetic factors that induce asthma, include allergens (eg. pollen, mold spore, animal hair, or dust), infections (eg. infections of viruses, bacteria, or mold inducing airway inflammation), temperature changes, drugs (eg. β-adrenergic antagonist or aspirin), some edible coloring, exercise, emotion, and other factors such as paint, perfume, cigarettes, air pollution, menstrual change, or gastro esophageal reflux diseases.

Since a complex disease trait is related to many genetic and non-genetic factors, patients suffering from the complex disease trait would have different symptoms, which may be due to differences in individuals, environments, ages, etiogenic factors, and types of the disease. So far, there are no standard criteria in diagnosing a complex disease trait, such as asthma (Britton J and Lewis S, Objective measures and the diagnosis of asthma. BMJ 1998;317:227-228; Talor D R, Making the diagnosis of asthma. BMJ 1997;315:4-5). Some standard diagnosing criteria, even though established, still fail to identify a complex disease trait and thus cannot be clinically used. Most physicians identify a complex disease trait by using a combination of history taking, physical examinations, lab examinations and/or radiodiagnostics. However, such a diagnosis method is not reliable due to the lack of overall consideration or experiences. Some complex disease traits usually cannot be identified because the symptoms of the complex disease traits would be mistaken for other diseases.

It is believed that history taking is not an objective index because it is difficult for children or the aged to remember or describe the symptoms. Also, physicians sometimes cannot make correct diagnoses because patients describe the symptoms in different ways.

There have been some studies on the diagnosis of a complex disease trait based on genetic testing (Quinzii C et al., Predictive genetic testing-new possibilities in determination of risk of complex diseases. CMJ 2001;42(4):458-462; Joos L and Standford A J, Genotype predictors of response to asthma medications. Current opinion in pulmonary medicine 2002;8:9-15; Brutsche M H et al., Array-based diagnostic gene-expression score for atopy and asthma. J Allergy Clin Immunol 2002;109:271-273; Sheppard D, Uses of expression microarrays in studies of pulmonary fibrosis, asthma, acute lung injury, and emphysema. Chest 2002;121(3 Suppl):21S-25S). The studies all focused on the few genes related to the complex disease traits. None of the above studies disclosed a correlation between multiple genes. Brutsche M H et al. provided a score, referred to as the “Composite Atopy Gene Expression (CAGE)”, for diagnosis of atopy and asthma. The CAGE represents an overall difference in the expression of 10 genes between the patient and the “normal people”. However, the CAGE may not be a good index for diagnosis since different genes function in different ways with various activation levels, and in addition, questions have also been raised regarding the definition of “normal people.”

Therefore, a scientific, quantitative, and rapid process for diagnosis of a complex disease trait is desired.

### SUMMARY OF THE INVENTION

An object of the invention is to provide a process of creating an index for diagnosis and/or prognosis of a complex disease trait in a subject, which comprises the steps of:

(a) detecting expression values of more than one gene selected to be related to the complex disease trait in said subject; and

(b) calculating the expression values using a correlation formula to obtain an index representing the possibility and/or severity of the subject suffering from the complex disease trait;

wherein the correlation formula in step (b) is obtained by a method comprising the steps of:

(i) estimating the condition scores of a group of patients suffering from the complex disease trait by history taking, physical examinations, lab examinations, and radiodiagnostics;

(ii) detecting expression values of the genes selected to be related to the complex disease trait of the patients; and

(iii) performing statistical analyses and obtaining a correlation formula based on the regression of the condition scores and the expression values of the patients obtained from steps (i) and (ii).

Another object of the invention is to provide a process of creating an asthma index for diagnosis and/or prognosis of asthma in a subject, which comprises the steps of:

(a) detecting expression values of more than one gene selected to be related to asthma in said subject; and

(b) calculating the expression values using a correlation formula to obtain an asthma index representing the possibility and/or severity of the subject suffering from asthma;

wherein the correlation formula in step (b) is obtained by a method comprising the steps of:

(i) estimating the condition scores of a group of patients suffering from asthma by history taking, physical examinations, lab examinations, and radiodiagnostics;

(ii) detecting expression values of the genes selected to be related to asthma of the patients; and

(iii) performing statistical analyses and obtaining a correlation formula based on the regression of the condition scores and the expression values of the patients obtained from steps (i) and (ii).

### DETAILED DESCRIPTION OF THE INVENTION

The present invention provides a process of creating an index for diagnosis and/or prognosis of a complex disease trait in a subject, comprising the steps of:

(a) detecting expression values of more than one gene selected to be related to the complex disease trait in said subject; and

(b) calculating the expression values using a correlation formula to obtain an index representing the possibility and/or severity of the subject suffering from the complex disease trait;

wherein the correlation formula in step (b) is obtained by a method comprising the steps of:

(i) estimating the condition scores of a group of patients suffering from the complex disease trait by history taking, physical examinations, lab examinations, and radiodiagnostics;

(ii) detecting expression values of the genes selected to be related to the complex disease trait of the patients; and

(iii) performing statistical analyses and obtaining a correlation formula based on the regression of the condition scores and the expression values of the patients obtained from steps (i) and (ii).

As used herein, the term “complex disease trait,” also known as a multifactorial disease, refers to a disease related to multiple genes, non-genetic factors, and the interaction between the multiple genes and non-genetic factors. A complex disease trait normally has polymorphous symptoms, and is usually mistaken for other diseases. The complex disease trait includes, but is not limited to, asthma, type 1 diabetic mellitus, rheumatic arthritis, system lupus erythematosus, ankylosing spondylitis, psoriasis or schizophrenia. In a preferred embodiment of the invention, the complex disease trait is asthma or rheumatic arthritis. The most preferred embodiment of the invention is asthma.

The term “index” used herein refers to a value representing the possibility and/or severity of the subject suffering from a disease or a condition. The term “condition score” used herein refers to a criterion or some criteria or their combination, for diagnosis and/or prognosis of a complex disease trait, such as symptoms felt by patients, sign tests by physicians, laboratory data, radiology finding and/or family histories, data combining history taking, physical examinations, lab examinations or radiodiagnostics. Any well established or newly defined condition scores for diagnosis of a complex disease trait can be used in the invention. In a preferred embodiment of the invention, asthma score referring to a combined estimate of asthma severity, medicine score referring to a frequency of medicine taken by patients, steroid score referring to a frequency of steroid drugs taken by patients, forced expiratory volume in 1 second (FEV₁), peak expiratory flow rate (PEFR), forced vital capacity (FVC), IgE amount, antigen specific to IgE, eosinophil, and eosinophil cationic protein (ECP) amount can be used as condition scores for diagnosis of asthma.

As used herein, the “genes selected to be related to a complex disease trait” refer to the genes or gene families, which are proved or supposed to be related to the complex disease trait. The genes include, but are not limited to, the genes directly or indirectly regulating the activation and/or degradation of cell expression, which is related to the complex disease trait, and the genes encoding the proteins directly or indirectly controlling all physiological reactions including intrinsic maintenance and responses to extrinsic changes. Preferably, there is more than one gene selected to be related to the complex disease trait. For example, the genes selected to be related to asthma are genes encoding cytokines, genes encoding receptors, genes encoding transcription factors, genes encoding signaling molecules, genes encoding chemokines, genes encoding adhesion molecules, or the combination.

According to the invention, the expression values of genes selected to be related to the complex disease trait can be detected by a gene chip or a polymerase chain reaction (PCR). The samples, which can be used for detection of the gene expression, comprise blood, serum, cell or tissue samples taken from a subject, preferably blood samples. The gene expression can be detected through hybridization with a target polynucleotide on a base complementation under strict conditions. In a preferred embodiment of the invention, multiple target polynucleotides are microarrayed on a solid or a chip in order to detect multiple gene expressions in one manipulation. Any detection methods for gene expression commonly used in the art can be used in the invention.

According to the invention, the correlation formula is obtained by performing statistical analyses and subsequent regressive analyses of the condition scores and the expression values of the patients. In a preferred embodiment of the invention, the statistical and regressive process is the Pearson correlation and multiples linear regression, which can be conducted through a commercial program such as the SPSS.

The accuracy of the diagnosis according to the invention depends on the genes selected and the number and diversity of the patients whose condition scores are to be collected for obtaining the correlation formula. It is preferable to choose as many genes as possible. However, not all genes are related to a complex disease trait. The number of the patients whose condition scores are to be collected for obtaining the correlation formula will also influence the accuracy. In theory, the accuracy of the diagnosis increases as the number of the patients increases. According to the invention, due to the diversity of patients, different correlation formulas can be obtained for different patient groups which are classified by sexes, ages, and/or living environments.

According to the present invention, physicians can obtain an index of a subject suspected to suffering from a complex disease train to determine if the subject suffers from the complex disease trait in a quick and objective way.

According to the invention, a process of creating an asthma index for diagnosis and/or prognosis of asthma in a subject, comprises the steps of:

(a) detecting expression values of more than one gene selected to be related to asthma in said subject; and

(b) calculating the expression values using a correlation formula to obtain an asthma index representing the possibility and/or severity of the subject suffering from asthma;

wherein the correlation formula in step (b) is obtained by a method comprising the steps of:

(i) estimating the condition scores of a group of patients suffering from asthma by history taking, physical examinations, lab examinations, and radiodiagnostics;

(ii) detecting expression values of the genes selected to be related to asthma of the patients; and

(iii) performing statistical analyses and obtaining a correlation formula based on the regression of the condition scores and the expression values of the patients obtained from steps (i) and (ii). The following Examples are given for the purpose of illustration only and are not intended to limit the scope of the present invention.

### EXAMPLE 1

Correlation Formula for Diagnosis of Asthma

Patients:

Fifty-two patients suffering from allergic asthma caused by dust mites were chosen based on the following criteria: (1) a raising total number of IgE in serum (more than 100 ku/mL); (2) a positive response of common allergens in skin test; (3) a raising number of CAP-specific IgE in serum (more than 2 ku/mL); and (4) a reversible raising lung function up to 15% after inhaling bronchodilator.

Estimation of Condition Scores of Asthma:

The patients suffering from asthma were identified by physician's history taking, physical examinations and lab examinations. The following condition scores for diagnosis of asthma of the patients were estimated based on the above-mentioned data: asthma score, medicine score, steroid score, forced expiratory volume in 1 second (FEV₁), peak expiratory flow rate (PEFR), forced vital capacity (FVC), IgE amount, antigen specific IgE, eosinophil, and eosinophil cationic protein (ECP) amount.

Preparation of Sample Polynucleotides: Blood samples were taken from the patients and collected in EDTA-contained tubes and then centrifuged at a speed of 2,500 rpm for 20 minutes to isolate a layer containing white blood cells. The white blood cells were washed by adding sterilized Phosphate buffer solution (PBS) into the layer containing white blood cells and then centrifuging it at a speed of 1,500 rpm for 10 minutes twice. The cells were then collected by a centrifugation at 4,000 g for 15 minutes at 4° C. Then, the cells were added with 1 mL of TRIZOL reagent and cracked by an oscillator. Then, the cells, after centrifugation, were mixed with 0.2 mL of CHCl₃ and oscillated again. The RNA and cell pellets were separated by a centrifugation at 14,000 g for 15 minutes at 4° C. Five hundreds μL isopropanol was added into the aliquot containing RNA, and mixed. The obtained mixture was kept at −20° C. for about 20 minutes. The pellet in the mixture was removed by a centrifugation at 14,000 g for 15 minutes at 4° C. After ethanol precipitation, the RNA in the mixture was dissolved in RNase-free water to obtain the sample polynucleotide. The concentration of the RNA was estimated (260 nm/280 nm).

Marker Labeling: Eight μL of the sample polynucleotides and 2 μL oligo poly-dT (12-18 mer, 0.1 μg/μL) were well mixed and kept at 70° C. for 10 minutes and then were cooled with ice for 2 minutes. The sample polynucleotides obtained were mixed with reverse transcription labeling mixture in dark and 3 μL Cy5-dUTP (1 mM), 2 μL SuperScript II (200U/μL), and Rnasin (1 μL). The mixture was incubated at 42° C. for 2 hours for reverse transcription, and the reaction was terminated by adding 1.5 μL 20 mM EDTA. The sample polynucleotides were degraded by adding 1.5 μL 500 mM NaOH and heated for 10 minutes. The NaOH retained in the sample polynucleotides was then neutralized by adding 1.5 μL 500 mM HCl, and excess Cy5 was removed by spinning in ProbeQuant G-50 Micro Column. All the sample nucleotides labeled with Cy5 were stored at −20° C.

Preparation of Target Polynucleotides: The genes chosen were amplified through polymerase chain reaction and then dissolved in spotting buffer as the target polynucleotides. After denaturing at 95° C. for 3 minutes, the target polynucleotides were attached to a glass carrier by ultra-violet rays using a spotting machine to form a chip for detection of gene expression.

Interactions Between Target Polynucleotides and The Sample Polynucleotides: The chip with the target polynucleotides was pretreated by n-methyl-pyrilidinone/succinic anhydride/sodium borate and 5×SSC/0.1% SDS/1% BSA to eliminate nonspecific hybridization by blocking active groups on the glass carrier. The sample polynucleotides labeled with Cy5 in hybridization buffer (50% formamide/0.2% SDS/10×SSC) were then denatured at 95° C. for 5 minutes and cooled. The sample polynucleotides were loaded to the chip. Hybridizations of the target polynucleotides and the sample polynucleotides were performed at 42° C. for 18 hours. Three solutions of 1×SSC/0.1% SDS, 0.1×SSC/0.1% SDS and 0.1×SSC were used to wash the samples and to remove the nucleotides which were non-specific to the target nucleotides or the nucleotides which were not hybridized.

Signals Detection: Gene expressions were detected and analyzed by scanning the chips using a fluorescence scanner and further quantified to obtain expression values. The fluorescent signals were quantified with GenePix™ Pro 3.0 (Axon Instruments, Inc.) and the backgrounds were then deduced, and then divided by the GAPDH (glyceraldehydes phosphate dehydrogenase, a house keeping gene). Mouse cDNA (ATBS) and plants DNA (RbCL) were both chosen as negative control.

Analysis: Each of the expression values was represented in a mean of duplicate. The Pearson correlation and multiples linear regression for each of the condition scores of asthma and the expression of each of the selected genes were conducted through the SPSS 8.01 statistical package.

The correlation of each of the condition scores of asthma and each of the gene expression values is listed in Table 1.

TABLE 1

| | | | | | Dp- | | |
|---|---|---|---|---|---|---|---|
| | Asthma | | Steroid | | specific | Eosinophil | |
| Gene Type | Score | FEV₁ | Score | IgE | IgE | Count | ECP |
| ACHE | −.281 | .224 | −.212 | −.073 | .029 | .194 | .181 |
| | (.006)** | (.066)* | (0.039)** | (.490) | (.780) | (.064)* | (.104) |
| CCR1 | −.110 | −.217 | .073 | −.108 | −.134 | −.022 | .023 |
| | (.289) | (.076)* | (.481) | (.304) | (.201) | (.838) | (.837) |
| CD31 | −.183 | −.197 | .015 | −.097 | −.047 | .088 | .128 |
| | (.076)* | (.107) | (.885) | (.356) | (.655) | (.405) | (.251) |
| Colony | −.039 | −.022 | −.032 | −.159 | −.160 | .014 | .147 |
| stimulating | (.707) | (.859) | (.757) | (.129) | (.126) | (.893) | (.188) |
| factor 3 | | | | | | | |
| GBP1 | .011 | .100 | −.155 | .095 | .028 | .069 | .201 |
| | (.919) | (.419) | (.134) | (.367) | (.793) | (.511) | (.071)* |
| IL12 receptor | −.178 | .170 | −.021 | .021 | .109 | .112 | .216 |
| beta 2 | (.085)* | (.167) | (.843) | (.846) | (.299) | (.287) | (.051)* |
| IL18 receptor | −.105 | .037 | −.095 | −.057 | .011 | .163 | .281 |
| | (.312) | (.766) | (.357) | (.591) | (.914) | (.121) | (.011)** |
| IRF4 | −.082 | .048 | −.054 | −.028 | .040 | .253 | .088 |
| | (.427) | (.699) | (.602) | (.793) | (.706) | (.015)** | (.432) |
| Metallothionein | −.276 | .006 | −.134 | −.025 | .014 | .088 | .290 |
| | (.007)** | (.961) | (.190) | (.816) | (.808) | (.406) | (.008)** |
| MUC2 | −.019 | .018 | −.134 | .027 | .032 | .247 | .260 |
| | (.065)* | (.886) | (.197) | (.795) | (.758) | (.018)** | (.019)** |
| SCYA4 | .229 | .282 | −.001 | −.180 | −.224 | .186 | −.042 |
| | (.026)** | (.020)** | (.991) | (.087)* | (.031)** | (.076)* | (.706) |
| STAT6 | −.171 | −.128 | −.128 | −.048 | .024 | .159 | .081 |
| | (.097)* | (.299) | (.217) | (.649) | (.031)** | (.129) | (.467) |
| ACHE_2 | −.118 | .048 | −.159 | .036 | .009 | .233 | .116 |
| | (.255) | (.698) | (.125) | (.737) | (.935) | (.026)** | (.299) |
| CCR3 | −.168 | −.162 | −.106 | −.045 | −.023 | .076 | .091 |
| | (.104) | (.187) | (.308) | (.668) | (.829) | (.473) | (.416) |
| CD34 | −.020 | −.039 | .080 | −.097 | −.116 | .048 | .201 |
| | (.851) | (.749) | (.443) | (.359) | (.270) | (.650) | (.071)* |
| CXCR3 | −.004 | .174 | .004 | .023 | −.032 | .158 | .082 |
| (GPR9) | (.967) | (.155) | (.972) | (.828) | (.760) | (.132) | (.463) |
| GBP2 | .240 | .242 | −.029 | −.031 | −.111 | .144 | −.001 |
| | (.019)** | (.046)** | (.782) | (.768) | (.289) | (.172) | (.991) |
| IL12 receptor | −.309 | .093 | −.214 | .014 | .055 | .180 | .040 |
| beta 2_2 | (.002)** | (.451) | (.037)** | (.894) | (.602) | (.086)* | (.772) |
| IL4 | .174 | .214 | .005 | −.098 | −.190 | .191 | .051 |
| | (.092)* | (.080)* | (.963) | (.355) | (.068)* | (.068)* | (.649) |
| IRF4_2 | −.286 | −.101 | −.178 | .102 | .106 | .165 | .125 |
| | (.005)** | (.411) | (.085)* | (.335) | (.312) | (.117) | (.265) |
| Metallothionein | −.385 | −.114 | −.238 | .096 | .061 | .107 | .246 |
| _2 | (.000)** | (.355) | (.020)** | (.362) | (.559) | (.310) | (.026)** |
| MUC5AC | −.110 | −.145 | −.077 | −.023 | −.068 | .236 | .258 |
| | (.289) | (.236) | (.457) | (.875) | (.516) | (.023)** | (.019)** |
| Selection L | −.266 | .061 | −.337 | .038 | .116 | .156 | .183 |
| | (.009)** | (.620) | (.001)** | (.716) | (.267) | (.138) | (.101) |
| TBXA2R | −.053 | −.088 | −.114 | .056 | .006 | .092 | .259 |
| | (.611) | (.474) | (.271) | (.593) | (.955) | (.382) | (.019)** |
| Adenylate | .171 | .137 | .053 | −.180 | −.180 | .032 | −.121 |
| cyclase 1 | (.098)* | (.266) | (.611) | (.086)* | (.085)* | (.209) | (.277) |
| CCR5 | −.243 | −.088 | −.055 | −.062 | −.001 | .229 | .143 |
| | (.018)** | (.477) | (.595) | (.554) | (.990) | (.028)** | (.200) |
| CD38 | −.210 | −.047 | −.066 | .042 | .028 | .116 | .030 |
| | (.041)** | (.705) | (.525) | (.692) | (.792) | (.269) | (.791) |
| EGR2 | −.058 | −.131 | −.055 | −.015 | −.096 | .035 | .048 |
| | (.575) | (.286) | (.595) | (.888) | (.360) | (.739) | (.666) |
| HOXA1 | −.146 | .014 | −.054 | −.022 | −.081 | .067 | .206 |
| | (.157) | (.913) | (.602) | (.838) | (.441) | (.523) | (.043)** |
| IL 13 | −.295 | .056 | −.244 | .229 | .155 | .190 | −.051 |
| | (.004)** | (.650) | (.017)** | (.028)** | (.138) | (.069)* | (.647) |
| IL4 receptor | −.476 | −.017 | −.112 | .026 | .046 | .167 | .190 |
| alpha | (.000)** | (.891) | (.282) | (.802) | (.664) | (.111) | (.087)* |
| ITGA 6 | −.108 | .053 | −.208 | .082 | .017 | .078 | .155 |
| | (.296) | (.674) | (.043)** | (.436) | (.868) | (.460) | (.164) |
| Metallothionein | .171 | .250 | −.025 | −.042 | −.114 | .266 | .011 |
| | (.098)* | (.040)** | (.809) | (.682) | (.278) | (.010)** | (.920) |
| PDE4B | −.074 | −.176 | −.158 | .009 | .000 | .129 | .290 |
| | (.476) | (.150) | (.125) | (.935) | (.998) | (.219) | (.008)** |
| SLAM | −.214 | −.149 | −.194 | .093 | .025 | .112 | .104 |
| | (.037)** | (.225) | (.059)* | (.378) | (.815) | (.288) | (.352) |
| TBSA2R_2 | .202 | .268 | −.019 | −.108 | −.166 | .201 | −.024 |
| | (.050)** | (.027)** | (.854) | (.305) | (.111) | (.054)* | (.827) |
| Adenylate | −.123 | −.013 | −.202 | −.066 | −.033 | .344 | .246 |
| cyclase 1_2 | (.234) | (.919) | (.050)** | (.535) | (.756) | (.001)** | (.026)** |
| CCR7 | −.380 | −.034 | −.261 | .139 | .160 | .256 | .169 |
| | (.000)** | (.782) | (.011)** | (.186) | (.126) | (.014)** | (.128) |
| CD69 | −.039 | .147 | .104 | .051 | .055 | .234 | .158 |
| | (.709) | (.230) | (.314) | (.632) | (.600) | (.025)** | (.155) |
| Eotaxin | −.050 | .055 | −.001 | −.089 | −.112 | .079 | −.014 |
| | (.631) | (.658) | (.991) | (.398) | (.285) | (.456) | (.897) |
| HOXA1_2 | −.114 | .020 | −.117 | −.172 | −.160 | .040 | .198 |
| | (.272) | (.873) | (.258) | (.102) | (.120) | (.705) | (.074)* |
| IL 15 | .312 | .137 | .017 | −.055 | −.099 | .119 | −.017 |
| | (.002)** | (.264) | (.872) | (.603) | (.346) | (.259) | (.879) |
| IL 5 receptor | −.194 | −.052 | −.238 | .044 | −.036 | .073 | .190 |
| alpha | (.060)* | (.675) | (.020)** | (.678) | (.733) | (.488) | (.087)* |
| ITGB7 | −.409 | .108 | −.306 | .016 | .033 | .140 | .088 |
| | (.000)** | (.379) | (.003)** | (.881) | (.754) | (.182) | (.430) |
| MIG | −.101 | −.023 | −.066 | −.067 | −.014 | .150 | .287 |
| | (.330) | (.851) | (.526) | (.527) | (.896) | (.155) | (.009)** |
| PDPK | .277 | .077 | .001 | −.102 | −.114 | .213 | .005 |
| | (.006)** | (.534) | (.995) | (.334) | (.279) | (.041)** | (.967) |
| STAT1 | −.294 | −.063 | −.269 | .134 | .145 | .185 | .126 |
| | (.004)** | (.611) | (.008)** | (.204) | (.105) | (.078)* | (.218) |
| TBXA2R_3 | −.044 | −.034 | .017 | −.011 | −.028 | .052 | .172 |
| | (.670) | (.781) | (.872) | (.915) | (.790) | (.623) | (.123) |
| Adenylate | .287 | .066 | .109 | −.120 | −.145 | .141 | .014 |
| cyclase 1_3 | (.005)** | (.591) | (.292) | (.254) | (.165) | (.180) | (.900) |
| CD2 | .140 | .284 | −.016 | −.183 | −.171 | .173 | .020 |
| | (.175) | (.019) | (.881) | (.082)* | (.101) | (.099)* | (.860) |
| CD97 | −.010 | .153 | .038 | .062 | .024 | .211 | .055 |
| | (.925) | (.213) | (.716) | (.558) | (.822) | (.044)** | (.627) |
| ETS1 | −.039 | −.075 | −.028 | −.140 | −.043 | .082 | .113 |
| | (.711) | (.544) | (.785) | (.182) | (.084)* | (.436) | (.313) |
| ICAM1 | −.098 | −.207 | −.064 | −.041 | −.066 | .122 | .088 |
| | (.346) | (.091)* | (.540) | (.701) | (.528) | (.248) | (.431) |
| IL15_2 | −.056 | −.170 | .017 | −.056 | −.032 | .070 | .233 |
| | (.587) | (.166) | (.872) | (.598) | (.758) | (.505) | (.035)** |
| Il 5 receptor | −.196 | .149 | −.088 | .076 | .030 | .074 | .307 |
| alpha_2 | (.057)* | (.226) | (.395) | (.472) | (.772) | (.483) | (.005)** |
| LAMR1 | −.346 | .190 | −.172 | .156 | .172 | .239 | .216 |
| | (.001)** | (.121) | (.096)* | (.137) | (.098)* | (.022)** | (.051) |
| MUC1 | −.032 | −.059 | .070 | −.106 | −.067 | .191 | .115 |
| | (.761) | (.632) | (.500) | (.315) | (.522) | (.069)* | (.302) |
| PRKG1 | .048 | −.077 | .091 | .051 | −.058 | .115 | .211 |
| | (.646) | (.533) | (.382) | (.632) | (.582) | (.276) | (.053)* |
| STAT2 | −.085 | −.022 | −.050 | −.133 | −.119 | .112 | .015 |
| | (.412) | (.861) | (.632) | (.207) | (.255) | (.289) | (.892) |
| Terminal | −.071 | −.025 | −.045 | .063 | .047 | .098 | .115 |
| transferase | (.495) | (.840) | (.663) | (.552) | (.653) | (.353) | (.304) |
| ADRB2 | −.157 | −.054 | −.035 | −.131 | −.100 | .020 | .275 |
| | (.130) | (.660) | (.738) | (.214) | (.314) | (.853) | (.012)** |
| CD26 | −.070 | −.038 | .044 | −.117 | −.132 | .109 | .221 |
| | (.501) | (.756) | (.673) | (.269) | (.208) | (.300) | (.046)** |
| CDH3 | −.227 | .131 | −.087 | −.034 | −.032 | .139 | .111 |
| | (.027)** | (.288) | (.401) | (.750) | (.760) | (.185) | (.320) |
| ETS1_2 | .026 | −.013 | −.052 | .021 | −.148 | −.016 | .257 |
| | (.801) | (.916) | (.615) | (.841) | (.157) | (.881) | (.020)** |
| ICAM2 | −.403 | −.111 | −.247 | .099 | .051 | −.027 | .104 |
| | (.000)** | (.369) | (.016)** | (.347) | (.630) | (.798) | (.354) |
| Il 15_3 | .255 | .372 | .049 | −.074 | −.174 | .202 | .165 |
| | (.013)** | (.000)** | (.636) | (.484) | (.095)* | (.053)* | (.139) |
| Il5 receptor | −.100 | −.013 | .024 | .047 | .133 | .108 | .106 |
| alpha_3 | (.334) | (.914) | (.817) | (.655) | (.203) | (.305) | (.342) |
| Lymphotactin | −.074 | −.012 | −.045 | .030 | .035 | .051 | .109 |
| beta | (.485) | (.924) | (.665) | (.774) | (.203) | (.627) | (.330) |
| MUC2_2 | −.238 | −.050 | −.091 | .066 | .084 | .173 | .212 |
| | (.020)** | (.083)* | (.381) | (.534) | (.425) | (.100) | (.007)** |
| PTGER2 | .020 | −.238 | .064 | −.104 | −.185 | .045 | .293 |
| | (.846) | (.050)** | (.537) | (.323) | (.076)* | (.672) | (.007)** |
| STAT4 | −.122 | −.057 | −.061 | .046 | .060 | .137 | .255 |
| | (.239) | (.646) | (.560) | (.664) | (.569) | (.192) | (.021)** |
| Aldehyde | −.206 | −.035 | −.130 | −.056 | −.062 | −.026 | .209 |
| dehydrogenase | (.045)** | (.780) | (.211) | (.593) | (.553) | (.809) | (.059)* |
| 1 | | | | | | | |
| CD30 | −.199 | −.070 | −.239 | −.073 | .009 | .135 | .059 |
| | (.053)* | (.572) | (.020)** | (.488) | (.933) | (.200) | (.599) |
| CEBPB | .002 | .214 | .073 | −.130 | −.133 | .266 | .124 |
| | (.986) | (.079)* | (.481) | (.217) | (.204) | (.010)** | (.269) |
| GATA1 | .233 | .158 | .043 | .006 | −.082 | .194 | .045 |
| | (.023)** | (.987) | (.682) | (.955) | (.432) | (.069)* | (.691) |
| Interferon 1 | .128 | −.001 | .012 | .042 | −.035 | .079 | .100 |
| | (.218) | (.993) | (.908) | (.693) | (.738) | (.452) | (.370) |
| IL 15_4 | −.115 | .018 | −.163 | −.012 | −.089 | −.001 | .226 |
| | (.267) | (.885) | (.114) | (.913) | (.396) | (.989) | (.041)** |
| IL 6 | .318 | .203 | −.004 | −.077 | −.112 | .142 | −.082 |
| | (.002)** | (.097)* | (.968) | (.467) | (.283) | (.176) | (.463) |
| MCP−3 | −.121 | .075 | −.007 | .057 | .078 | .252 | .112 |
| | (.245) | (.543) | (.949) | (.592) | (.456) | (.015)** | (.316) |
| MUC2_3 | −.108 | −.147 | −.035 | −.077 | −.138 | .034 | .215 |
| | (.295) | (.232) | (.733) | (.466) | (.187) | (.748) | (.053)* |
| RANTES | .204 | .330 | .021 | −.129 | −.124 | .225 | .026 |
| | (.047)** | (.006)** | (.840) | (.220) | (.238) | (.031)** | (.815) |
| STAT4_2 | −.124 | −.094 | −.089 | .121 | .121 | .003 | .158 |
| | (.230) | (.452) | (.392) | (.252) | (.246) | (.979) | (.156) |
| ANXA3 | .234 | .193 | .050 | −.094 | −.098 | .158 | .001 |
| | (.023)** | (.114) | (.628) | (.374) | (.348) | (.132) | (.991) |
| CD30_2 | −.007 | .170 | .108 | .016 | −.025 | .187 | .144 |
| | (.944) | (.166) | (.298) | (.883) | (.814) | (.074)* | (.198) |
| c-fos | .333 | −.102 | .080 | .011 | −.027 | .198 | .153 |
| | (.001)** | (.406) | (.442) | (.914) | (.794) | (.059)* | (.170) |
| GATA3 | −.222 | −.032 | −.092 | .033 | .023 | .192 | .266 |
| | (.030)** | (.795) | (.376) | (.755) | (.824) | (.067)* | (.016)** |
| IL 10 | −.062 | .022 | −.144 | −.079 | −.119 | −.002 | .128 |
| | (.549) | (.860) | (.164) | (.456) | (.257) | (.986) | (.251) |
| IL 18 | .189 | .259 | −.016 | −.058 | −.123 | .227 | −.001 |
| | (.066)* | (.033)** | (.881) | (.581) | (.240) | (.029)** | (.991) |
| IRF4_3 | −.139 | −.095 | −.140 | .011 | −.016 | .074 | .122 |
| | (.179) | (.440) | (.177) | (.918) | (.880) | (.486) | (.274) |
| Metallothionein | −.408 | −.059 | −.201 | .053 | .015 | .054 | .219 |
| _4 | (.000)** | (.631) | (.051)* | (.615) | (.884) | (.612) | (.048)** |
| MUC2_4 | −.218 | −.228 | −.092 | −.033 | .054 | .162 | .203 |
| | (.034) | (.062)* | (.374) | (.757) | (.608) | (.122) | (.068)* |
| SCYA17 | .307 | .291 | .026 | −.220 | −.218 | .171 | −.049 |
| | (.002)** | (.016)** | (.804) | (.035)** | (.036)** | (.104) | (.661) |
| STAT4_3 | −.156 | −.113 | .001 | .028 | .065 | .006 | .101 |
| | (.131) | (.357) | (.991) | (.788) | (.536) | (.958) | (.368) |

Note. **P < 0.05; *P < 0.1

The parameters of the asthma-related gene expression for creating an asthma index are listed in Table 2:

TABLE 2

| | Gene Type | Parameter | P Value | R square |
|---|---|---|---|---|
| Asthma | | | 3.93 | |
| Score | | | | 0.706 |
| | | | (0.000)** | |
| | ACHE | −0.140 | | |
| | CD31 | 0.866 | | |
| | IL12 receptor beta 2 | −0.127 | | |
| | Metallothionein | 1.711 | | |
| | MUC2 | −.108 | | |
| | SCYA4 | 3.260 | | |
| | STAT6 | 0.725 | | |
| | GBP2 | 4.478 | | |
| | IL4 | −5.707 | | |
| | IRF4_2 | −0.457 | | |
| | SELECTIN_L | 1.560 | | |
| | Adenylate cyclase1 | −3.660 | | |
| | CCR5 | 1.788 | | |
| | CD38 | 0.034 | | |
| | IL13 | 2.778 | | |
| | IL4 receptor alpha | −6.390 | | |
| | SLAM | 0.513 | | |
| | TBXA2R_2 | −4.276 | | |
| | CCR7 | −1.519 | | |
| | IL15 | −0.218 | | |
| | IL5 receptor alpha | −0.893 | | |
| | ITGB7 | −2.400 | | |
| | PDPK | 2.762 | | |
| | STAT1 | −2.458 | | |
| | LAMR1 | 2.920 | | |
| | CDH3 | −0.470 | | |
| | ICAM2 | 0.066 | | |
| | aldehyde | −2.534 | | |
| | dehydrogenase1 | | | |
| | CD30 | −0.146 | | |
| | GATA1 | −2.006 | | |
| | IL6 | −3.633 | | |
| | RANTES | −4.335 | | |
| | ANXA3 | 2.871 | | |
| | C-FOS | 1.567 | | |
| | GATA3 | 1.149 | | |
| | SCYA17 | 10.946 | | |

In addition, the value of FEV % and the parameters of the asthma-related gene expression are listed in Table 3:

TABLE 3

| Regression | | | | |
|---|---|---|---|---|
| Model | Gene Type | Parameter | P Value | R square |
| | SCYA4 | 3.594 | | |
| | GBP2 | 6.037 | | |
| | IL4 | −9.400 | | |
| | IL15-3 | 2.576 | | |
| | | | 34.75 | |
| FEV% | PTGER2 | 1.945 | | 0.814 |
| | | | (0.000)** | |
| | IL6 | 1.189 | | |
| | RANTES | 4.093 | | |
| | IL18 | −8.355 | | |
| | MUC2_4 | 0.191 | | |

A correlation formula based on asthma score is obtained by using the data of Table 2 and the Pearson correlation and multiples linear regression:

$$\begin{matrix}Asthma Index = - 0.140 ( ACHE ) + 0.866 ( CD 31 ) - \\ 0.127 ( IL 2 receptor beta 2 ) + 1.711 ( metallothionein ) - \\ 0.108 ( MUC 2 ) + 3.260 ( SCYA 4 ) + 0.725 ( STAT 6 ) + 4.748 ( GBP 2 ) - \\ 5.707 ( IL 4 ) - 0.457 ( IRF4\_ 2 ) + 1.560 ( SELECTIN\_L ) - \\ 3.660 ( adenylate cyclase l ) + 0.719 ( CCR 5 ) + 0.034 ( CD 38 ) + \\ 2.778 ( IL1 \; 3 ) - 6.390 ( IL 4 receptor alpha ) + 0.513 ( SLAM ) - \\ 4.276 ( TBXA2R\_ 2 ) - 1.519 ( CCR7 ) - 0.218 ( IL15 ) - \\ 0.893 ( IL 5 receptor alpha ) - 2.400 ( ITGB7 ) + 2.762 ( PDPK ) - \\ 2.458 ( STAT1 ) + 2.920 ( LAMR 1 ) - 0.470 ( CDH 3 ) + 0.066 ( ICAM 2 ) - \\ 2.534 ( aldehyde dehydrogenase l ) - 0.146 ( CD 30 ) - 2.006 ( GATA 1 ) - \\ 3.633 ( IL 6 ) - 4.355 ( RANTES ) + 2.871 ( ANXA 3 ) + 1.567 ( C \text{-} FOS ) + \\ 1.149 ( GATA 3 ) + 10.946 ( SCYA 17 ) .\end{matrix}$$

A correlation formula based on FEV % is obtained by using the data of Table 3:

$$\begin{matrix}Asthma Index Based On FEV \% = 3.594 ( SCYA 4 ) + 6.037 ( GBP 2 ) - \\ 9.400 ( IL 4 ) + 2.576 ( IL 15 \text{-} 3 ) + 1.945 ( PTGER 2 ) + 1.189 ( IL 6 ) + \\ 4.093 ( RANTES ) - 8.355 ( IL 1 ) + 0.191 ( MUC2\_ 4 ) .\end{matrix}$$

### EXAMPLE 2

Diagnosis by Asthma Index

Blood samples were taken from a group of the patients and the polynucleotides contained in the samples were labeled with Cy5 according to the methodology as described in Example 1. The samples were detected with the chips as obtained in Example 1. The expression values of the genes selected to be related to asthma were quantified and normalized. The asthma index of each subject was obtained using the expression values based on the correlation formulas of Condition Asthma Index Based On Asthma Score and Asthma Index Based On FEV % as obtained above. The asthma indexes obtained from the correlating formula based on asthma score and on FEV % were 70.6% (P<0.05) and 81.4% (P<0.05), respectively.

While embodiments of the present invention have been illustrated and described, various modifications and improvements can be made by persons skilled in the art. The embodiments of the present invention are therefore described in an illustrative but not restrictive sense. It is intended that the present invention is not limited to the particular forms as illustrated, and that all the modifications not departing from the spirit and scope of the present invention are within the scope as defined in the appended claims.

What is claimed is:

1. A process of creating an index for diagnosis and/or prognosis of a complex disease trait in a subject, which comprises the steps of:
(a) detecting expression values of more than one gene selected to be related to the complex disease trait in said subject; and
(b) calculating the expression values using a correlation formula to obtain an index representing the possibility and/or severity of the subject suffering from the complex disease trait;
wherein the correlation formula in step (b) is obtained by a method comprising the steps of:
(i) estimating the condition scores of a group of patients suffering from the complex disease trait by history taking, physical examinations, lab examinations, and radiodiagnostics;
(ii) detecting expression values of the genes selected to be related to the complex disease trait of the patients; and
(iii) performing statistical analyses and obtaining a correlation formula based on the regression of the condition scores and the expression values of the patients obtained from steps (i) and (ii).

2. The process according to claim 1, wherein the expression values of the genes in step (b) can be determined by a chip or a polymerase chain reaction.

3. The process according to claim 2, wherein the genes to be tested for expression are obtained from blood samples of the subjects.

4. The process according to claim 2, wherein the expression value of a gene in step (ii) is determined by a chip or a polymerase chain reaction.

5. The process according to claim 1, wherein the statistic analysis and regression process of the condition scores and the expression values in step (iii) is the Pearson correlation and multiple linear regression.

6. A process of obtaining an asthma index for diagnosis and/or prognosis of asthma in a subject, which comprises the steps of:
(a) detecting expression values of more than one gene selected to be related to asthma in said subject; and
(b) calculating the expression values using a correlation formula to obtain an asthma index representing the possibility and/or severity of the subject suffering from asthma;
wherein the correlation formula in step (b) is obtained by a method comprising the steps of:
(i) estimating the condition scores of a group of patients suffering from asthma by history taking, physical examinations, lab examinations, and radiodiagnostics;
(ii) detecting expression values of the genes selected to be related to asthma of the patients; and
(iii) performing statistical analyses and obtaining a correlation formula based on the regression of the condition scores and the expression values of the patients obtained from steps (i) and (ii).

7. The process according to claim 6, wherein the genes to be tested for expression are obtained from blood samples of the subject or the patients.

8. The process according to claim 6, wherein the expression values of the genes in step (b) is determined by a chip or a polymerase chain reaction.

9. The process according to claim 6, wherein the expression value of a gene in step (ii) is determined by a chip or a polymerase chain reaction.

10. The process according to claim 6, wherein the statistic analysis and regression process for the condition scores and the expression values in step (iii) is the Pearson correlation and multiple linear regression.

11. The process according to claim 6, wherein the genes selected to be related to asthma comprise genes encoding cytokines, genes encoding receptors, genes encoding transcription factors, genes encoding signaling molecules, genes encoding chemokines, genes encoding adhesion molecules or their combination.

12. The process according to claim 6, wherein the condition score is selected from the group consisting of asthma score, medicine score, steroid score, forced expiratory volume in 1 second (FEV₁), peak expiratory flow rate (PEFR), forced vital capacity (FVC), IgE amount, antigen specific IgE, eosinophil, eosinophil cationic protein (ECP) amount, and the their combination.
